(19) United States
(12) Patent Application Publication
(10) Pub. No.: US 2026/0058024 A1
(43) Pub. Date: Feb. 26, 2026

(54) MACHINE LEARNING SYSTEMS AND RELATED ASPECTS FOR GENERATING DISEASE MAPS OF POPULATIONS

(71) Applicant: ARIZONA BOARD OF REGENTS ON BEHALF OF ARIZONA STATE UNIVERSITY, SCOTTSDALE, AZ (US)

(72) Inventors: Neal Woodbury, Tempe, AZ (US); Alexander Taguchi, Cambridge, MA (US); Laimonas Kelbauskas, Chandler, AZ (US); Robayet Chowdhury, Richland, WA (US)

(21) Appl. No.: 19/099,528

(22) PCT Filed: Sep. 15, 2023
(86) PCT No.: PCT/US2023/074296

§ 371 Date: Jan. 29, 2025

Related U.S. Application Data
(60) Provisional application No. 63/377,976, filed on Sep. 30, 2022.

(51) Int. Cl. G16H 50/80; G06N 3/08

(52) U.S. Cl. CPC G16H 50/80; G06N 3/08

(57) ABSTRACT

Provided herein are computer-implemented methods of generating a disease map of a population. In some embodiments, the methods include applying a clustering algorithm to a set of weight and bias values of a trained electronic neural network to generate the disease map of the population. In some embodiments, the electronic neural network has been trained on training data that comprises representations of peptide sequence and binding value pair data sets obtained from reference subjects in the population in which a given peptide sequence and binding value pair data set comprises peptide sequence information and peptide binding values of antibodies to peptides that comprises the peptide sequence information. In some embodiments, the antibodies are from a sample obtained from a given reference subject in the population and are indicative of one or more disease states. Related systems, computer readable media, and additional methods are also provided.

### CROSS-REFERENCE TO RELATED APPLICATIONS

This application claims the benefit of and priority to U.S. Provisional Patent Application Ser. No. 63/377,976, filed Sep. 30, 2022, the disclosure of which is incorporated herein by reference.

### FIELD

This disclosure relates generally to machine learning, e.g., in the context of medical applications, such as pathology.

### BACKGROUND

From the point of view of chemical biology, the humoral immune response to infection represents a truly remarkable example of rapid directed molecular evolution. In a matter of days to weeks, high affinity, high specificity molecular recognition of a previously unknown target is developed, mediated by antibodies. This is far more than just passive in vivo panning of a molecular library. It is instead a very active process in which initially weakly binding ligands are identified in a very sparse representation of the total possible antibody sequence space and these are iteratively evolved to optimize both binding and specificity by orders of magnitude, a process mediated by B cells. The fact that this takes place on such a rapid timescale almost requires that the ascent from weak, less specific binding to strong specific binding can take place in a systematic, more or less continuous, fashion by changing small numbers of amino acids per round, in other words, that the topology of the amino acid sequence space involved in maturing an antibody binding sequence is locally smooth between some starting sequences and some optimized sequences. If this is in fact the case, it is not unreasonable to hypothesize that the converse might also be true: the amino acid sequence space of antigens or epitopes involved in an immune response might also be locally smooth relative to antibody binding. A locally smooth space should be predictable via interpolation and extrapolation; a sparse sampling of IgG binding to sequences in that sequence space should enable one to generate a quantitative relationship that predicts the IgG binding at other close-by sequences in the space not originally sampled, resulting in a predictive mathematical representation for the molecular recognition space of an immune response in terms of antigen sequence.

Accordingly, there is a need for additional models for use in disease diagnostics, including infectious disease detection, and other applications that demonstrate improved performance over currently available models.

### SUMMARY

The present disclosure provides, in certain aspects, an artificial intelligence (AI) system capable of generating disease maps of populations. In some aspects, for example, the present disclosure shows that by using the binding of antibodies in serum to molecular arrays, such as arrays of peptides, it is possible to identify known and unknown diseases in populations based on unsupervised clustering of the data, particularly after processing using machine learning algorithms relating chemical structure to antibody molecular recognition. In some embodiments, the methods and related systems are used to rapidly map disease prevalence for known diseases (e.g., those that have known positions on a given disease map) and unknown diseases (e.g., those that suddenly appear in new places on a given disease map). Exemplary applications of the methods and related systems of the present disclosure include in blood banks to scan for outliers, in congregate settings, such as nursing homes, to look for outbreaks, and in large-scale bio-surveillance systems to monitor epidemics and pandemics, among other applications. These and other aspects will be apparent upon a complete review of the present disclosure, including the accompanying figures.

According to various embodiments, a computer-implemented method of generating a disease map of a population is presented. The method includes: applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.

Various optional features of the above embodiments include the following. At least one of the disease states is known. At least one of the disease states is unknown. At least one of the disease states comprises an infectious disease state. The disease map comprises clusters of the disease states represented in a two or more dimensional space (e.g., about 3, about 4, about 5, about 10, about 25, about 50, about 100, or more dimensions). The clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and an HCS clustering algorithm, among other clustering algorithms that are optionally adapted for use with the methods and other aspects of the present disclosure. The set of weight and bias values is a final set of weight and bias values of the trained electronic neural network. Producing the peptide sequence and binding value pair data sets from samples obtained from the reference subjects in the population. Determining whether a test subject has at least one of the disease states using a peptide sequence and binding value pair data set obtained from the test subject and the trained electronic neural network and/or the disease map. Generating at least one therapy recommendation for the test subject based at least in part a determination that the test subject has the at least one of the disease states. Administering one or more therapies to the test subject based at least in part on the therapy recommendation for the test subject. Generating at least one iteration of the disease map at a time point that differs from a time point at which the disease map was generated. Monitoring an immune status measure of the population and/or an occurrence of a known disease state or an unknown disease state in the population using the disease map and the iteration of the disease map.

According to various embodiments, a system for generating a disease map of a population using an electronic neural network is presented. The system includes a processor; and a memory communicatively coupled to the processor, the memory storing instructions which, when executed on the processor, perform operations including: applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.

Various optional features of the above embodiments include the following. At least one of the disease states is known. At least one of the disease states is unknown. At least one of the disease states comprises an infectious disease state. The disease map comprises clusters of the disease states represented in a two or more dimensional space (e.g., about 3, about 4, about 5, about 10, about 25, about 50, about 100, or more dimensions). The clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and an HCS clustering algorithm, among other clustering algorithms that are optionally adapted for use with the methods and other aspects of the present disclosure. The set of weight and bias values is a final set of weight and bias values of the trained electronic neural network. The instructions which, when executed on the processor, further perform operations comprising: determining whether a test subject has at least one of the disease states using a peptide sequence and binding value pair data set obtained from the test subject and the trained electronic neural network and/or the disease map. The instructions which, when executed on the processor, further perform operations comprising: generating at least one therapy recommendation for the test subject based at least in part a determination that the test subject has the at least one of the disease states. The instructions which, when executed on the processor, further perform operations comprising: generating at least one iteration of the disease map at a time point that differs from a time point at which the disease map was generated. The instructions which, when executed on the processor, further perform operations comprising: monitoring an immune status measure of the population and/or an occurrence of a known disease state or an unknown disease state in the population using the disease map and the iteration of the disease map.

According to various embodiments, a computer readable media is presented. The computer readable media comprises non-transitory computer executable instructions which, when executed by at least one electronic processor, perform at least: applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.

Various optional features of the above embodiments include the following. At least one of the disease states is known. At least one of the disease states is unknown. At least one of the disease states comprises an infectious disease state. The disease map comprises clusters of the disease states represented in a two or more dimensional space. The clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and/or an HCS clustering algorithm. The set of weight and bias values is a final set of weight and bias values of the trained electronic neural network. The instructions which, when executed by the processor, further perform operations comprising: determining whether a test subject has at least one of the disease states using a peptide sequence and binding value pair data set obtained from the test subject and the trained electronic neural network and/or the disease map. The instructions which, when executed by the processor, further perform operations comprising: generating at least one therapy recommendation for the test subject based at least in part a determination that the test subject has the at least one of the disease states. The instructions which, when executed by the processor, further perform operations comprising: generating at least one iteration of the disease map at a time point that differs from a time point at which the disease map was generated. The instructions which, when executed by the processor, further perform operations comprising: monitoring an immune status measure of the population and/or an occurrence of a known disease state or an unknown disease state in the population using the disease map and the iteration of the disease map.

### DRAWINGS

The above and/or other aspects and advantages will become more apparent and more readily appreciated from the following detailed description of examples, taken in conjunction with the accompanying drawings, in which:

FIG. 1A depicts a process of training a machine learning system in accordance with an embodiment.

FIG. 1B depicts a machine learning system developed according to the process shown in FIG. 1A in accordance with an embodiment.

FIG. 2 is a schematic diagram of an exemplary system suitable for use with certain aspects disclosed herein.

FIGS. 3A-3E show cohort data and neural network characteristics. (A) Average binding intensity distributions of serum IgG binding to array peptides for the 6 different sample cohorts. For each cohort the log₁₀ of the average for each peptide sequence was used to create the distribution. (B) The loss function progression during neural network training. Bottom two traces: a neural network trained with properly matched sequences and associated binding values. Top two traces: training after scrambling the order of the sequences relative to the binding values. (C) A neural network (2 hidden layers with 350 nodes) was trained on 95% of the sequence/binding data from the 542 low CV samples in Table 1 simultaneously. The remaining 5% of the sequence/binding values (6,146 per sample×542 samples=˜3.3 million binding values) were held out as the test set. The scatter plot (dscatter) shows the values predicted by neural network (y-axis) vs. the corresponding measured values from the array (x-axis) for the test set only. (D) Comparison of predicted vs. measured correlation coefficients calculated either by fitting samples simultaneously, as in panel C, or one at a time. (E) The average predicted vs. measured correlation coefficient for cohort samples using the neural network model of panel C as a function of the number of peptide sequences used to train the network.

FIGS. 4A-4E depict various aspects of discriminating between cohorts. (A) The data from the original array was analyzed in three ways: 1) directly, 2) after training a neural network and predicting the values of the array sequences, 3) after projecting the trained neural network on a complete new set of sequences. Disease discrimination was then performed for each approach using multi-class classification or by statistically determining the number of significant peptides distinguishing each cohort comparison. (B) Multi-class classification based on a neural network (see text). Classification was performed 100 times for each dataset leaving out 20% of the samples (randomly chosen) each time. Diagonal lines: original measured array data. Cross-hatched lines: neural network model prediction of binding values for array peptide sequences. Dotted: neural network projected onto a randomized set of sequences of the same overall size, composition and length distribution as the array sequences. (C) Each array element is the number of array peptides with measured binding values that are significantly higher in the sample cohort on the Y-axis compared to the sample cohort on the X-axis. Significance is defined as a p-value less than 1/N in a T-test with 95% confidence (N=122,926 total peptides, thus significant peptides have a p-value<0.05/N=4.1×10⁻⁷). (D) As in (A) except that the neural network predicted binding values of the array peptides were used instead of the measured. The mean of 10 different neural network model training runs is shown; error in the mean is <=0.3. (E) The same as in (D) except predicted values for an in silico generated array of random peptide sequences with the same average composition and length as the peptides in the array were used. The mean of 10 different sequence sets and neural network runs is shown; error of the mean is <=0.4.

FIGS. 5A-5B show the effect of added noise on multiclass classification. Noise was added to each peptide in the sample using a randomly chosen value from a gaussian distribution centered at the log₁₀ of the measured value. The sigma of the distribution was varied between 0 and 1 (the binding, and thus sigma, is on a log scale). (A) The resulting distributions of binding values for each sigma value. Distributions were determined after mean normalizing the binding values for each peptide in a cohort and then including all peptide binding values in the distribution. (B) results of applying a multi-class classifier (as in FIG. 4B) to the data for measured binding values (dashed lines) and predicted binding values (solid lines) at each value of sigma. Each classification was repeated 100 times (noise at each level was randomly added 10 times and each of these were reclassified 10 times leaving out 20% of the samples as the test set).

FIGS. 6A-6C depict the classification accuracy for high CV samples. (A) neural network predicted vs. measured values for low CV data and (B) for high CV data. (C) multiclass classification of the high CV data. Diagonally lined, Cross-hatched and Dotted bars represent use of measured, predicted and projected data as in FIG. 4.

FIG. 7 show the unsupervised clustering of the neural network final weight matrix plus bias. A Matlab implementation of UMAP (Uniform Manifold Approximation and Projection) was used to reduce 351 values from the final weight matrix of the neural network and the bias for each sample to 2 component values which are plotted. Cohorts are color coded.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Antibody: As used herein, the term “antibody” refers to an immunoglobulin or an antigen-binding domain thereof. The term includes but is not limited to polyclonal, monoclonal, monospecific, polyspecific, non-specific, humanized, human, canonized, canine, felinized, feline, single-chain, chimeric, synthetic, recombinant, hybrid, mutated, grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG1, IgG₂, IgG₃, IgG₄, IgM, IgA₁, IgA₂, IgD, and IgE. By way of example, the light chain constant region can be kappa or lambda. The term “monoclonal antibody” refers to an antibody that displays a single binding specificity and affinity for a particular target, e.g., epitope.

Binding Intensity: As used herein, the term “binding intensity” or “binding affinity”, typically refers to a strength of non-covalent association between or among two or more entities.

Classifier: As used herein, “classifier” generally refers to algorithm computer code that receives, as input, test data and produces, as output, a classification of the input data as belonging to one or another class.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., peptide sequence and binding value pair data sets or the like).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial or electronic neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Peptide: As used herein, “peptide” refers to a sequence of 2-50 amino acids attached one to another by a peptide bond. These peptides may or may not be fragments of full proteins. Examples of peptides include KPLEEVLN, FLPFQQK etc.

Protein: As used herein, “protein” or “polypeptide” refers to a polymer of typically more than 50 amino acids attached to one another by a peptide bond. Examples of proteins include enzymes, hormones, antibodies, peptides, and fragments thereof.

Sample: As used herein, a “sample,” such as a biological sample, is a sample obtained from a subject. As used herein, biological samples include all clinical samples including, but not limited to, cells, tissues, and bodily fluids, such as saliva, tears, breath, and blood; derivatives and fractions of blood, such as filtrates, dried blood spots, serum, and plasma; extracted galls; biopsied or surgically removed tissue, including tissues that are, for example, unfixed, frozen, fixed in formalin and/or embedded in paraffin; milk; skin scrapes; nails, skin, hair; surface washings; urine; sputum; bile; bronchoalveolar fluid; pleural fluid, peritoneal fluid; cerebrospinal fluid; prostate fluid; pus; or bone marrow. In a particular example, a sample includes blood obtained from a subject, such as whole blood or serum. In another example, a sample includes cells collected using an oral rinse. The sample may be isolated from the subject and then directly utilized in a method for determining the presence or absence of antibodies, or alternatively, the sample may be isolated and then stored (e.g., frozen) for a period of time before being subjected to analysis.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., a known pathology, such as melanoma and/or the like).

System: As used herein, “system” in the context of analytical instrumentation refers a group of objects and/or devices that form a network for performing a desired objective.

Treat: As used herein the terms “treat”, “treated”, or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

Value: As used herein, “value” generally refers to an entry in a data set that can be anything that characterizes the feature to which the value refers. This includes, without limitation, numbers, words or phrases, symbols (e.g., + or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. INTRODUCTION

The present disclosure generally describes systems and methods for developing and implementing machine learning systems, including regressor and classifiers, configured to model correlations between peptide binding data and a variety of different conditions, including disease states. Conventionally, a machine learning system used for diagnostics and related applications is trained on data related to a single condition and, accordingly, to identify that specific condition on which the machine learning system has been trained. However, the systems and methods described herein differ in that the machine learning systems are trained on data (e.g., peptide data) that is associated with a number of different disease states or other conditions. Importantly, the disease states or other conditions on which the machine learning systems are trained need not even be particularly related to each other in any particular manner. By training the machine learning system on a data associated with a range of different disease states or other conditions, the machine learning systems' performances are even improved with respect to each individual condition.

In some embodiments, the systems and methods described herein can be used as part of or in connection with an assay and/or kit for diagnosing one or more disease states or other conditions. The assay and/or kit can include reagents, probes, buffers, antibodies or other agents that enhance the binding of a subject's antibodies to biomarkers, signal generating reagents (e.g., fluorescent, enzymatic, electrochemical reagents), or separation enhancing methods (e.g., electromagnetic particles, nanoparticles, or binding reagents) for the detection of a combination of two or more biomarkers indicative thereof. In some embodiments, the probe and the signal-generating reagent may be one in the same. Exemplary techniques of use in all of these methods are discussed below.

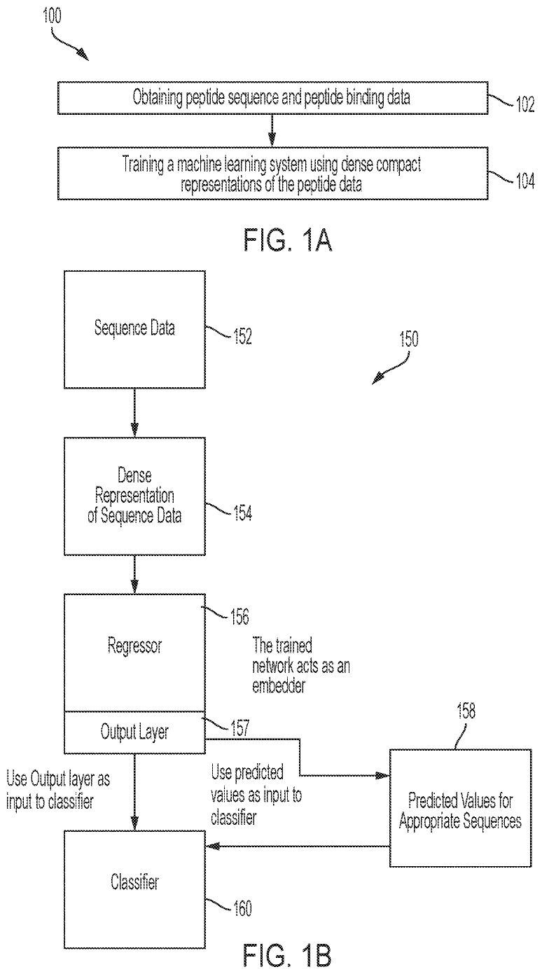

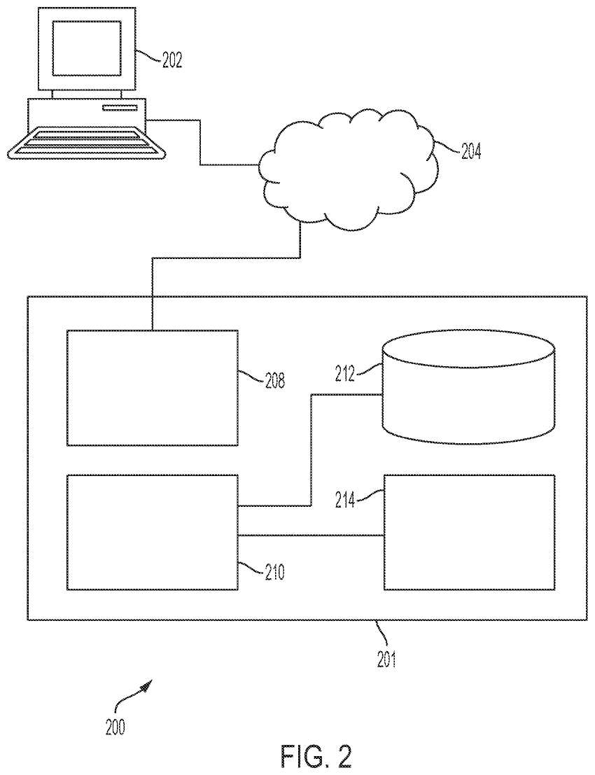

Described herein are systems and techniques for developing machine learning systems configured to identify a disease state or condition exhibited by data (e.g., peptide data) obtained from a sample from a patient and to generate related disease maps for populations. In one implementation, the systems and techniques described herein can be utilized to develop machine learning systems that model the sequence dependence of binding between peptide sequences (e.g., obtained via a peptide array) and the total serum IgG for each sample. In one embodiment, the systems and methods described can include the general process 100 illustrated in FIG. 1A. The process 100 can be executed by a computer system. For example, the process 100 can be embodied as instructions stored in a memory that, when executed by a processor, cause the processor and/or computer system to perform the illustrated steps. The process 100 can be utilized in a number of different applications, including medical diagnostics, bio-surveillance, or epitope discovery, as described below. Generally speaking, the process 100 involves the creation of a classification model (i.e., a classifier) that is configured to identify one or more disease states or conditions from peptide data obtained via a sample from a patient.

Accordingly, a computer system executing the process 100 can obtain 102 peptide data, such as peptide sequence data and/or peptide binding data. In one embodiment, the data can be obtained 102 via peptide arrays on one or more samples obtained from one or more patients (e.g., reference and/or test subjects), which may exhibit multiple disease states or conditions. The peptide data can be represented as, for example, a one-hot representation of the amino acids in each peptide sequence, i.e., the sequence can be represented as a sparse matrix of zeros and ones.

In some embodiments, the computer system can normalize the peptide binding values, for example, prior to training the machine learning system. In some embodiments, such normalization is not performed as part of the process 100. In an embodiment where the peptide data is represented via one-hot encoding, the computer system can multiply the obtained sparse matrix representing the peptide data by an encoder matrix that linearly transforms each amino acid into a dense compact representation, i.e., a real-valued vector. In one embodiment, the resulting matrix can then be flattened to form a real-valued vector representation for a peptide sequence, which is then utilized as the input to the first hidden layer of the neural network.

In some embodiments, the computer system can train 104 a machine learning system using dense compact representations of the of the peptide sequence data. The machine learning system can include one or more electronic neural networks, one or more support vector machines, and/or a variety of other machine learning models, for example. In some embodiments, the one or more electronic neural networks could include a feedforward neural network. In such embodiments, the electronic neural networks could be trained using back propagation, as is known in the technical field. In some embodiments, the machine learning system could be trained on a subset of the peptide sequence and binding paired data and the resulting machine learning system and/or individual machine learning models thereof could then be validated on the remaining subset of the peptide data, as is known in the technical field.

One embodiment of a machine learning system 150 developed using the process 100 is shown in FIG. 1B. In particular, the machine learning system 150 can include a regressor 156 that is trained on sequence data 152 and dense representations 154 of the sequence data, as described above. Once trained, the regressor 156 can function as an embedder for a classifier 160. In one embodiment, the classifier 160 could include a support vector machine. In some embodiments, classifier 160 comprises an electronic neural network. In particular, the output layer 157 and/or the predicted values 158 of the regressor 156 can be provided either individually or in combination with each other as input to a classifier 160, which then makes a classification on the provided input. In some embodiments, the input to the classifier 160 could include the predicted values 158 generated by the regressor 156 from a set of peptide sequences that between them provide differentiation between disease states. In some embodiments, the input to the classifier 160 could include the columns of the final output matrix of the regressor 156 itself, which contain a condensed version of the antibody profile information from each of the samples. In some embodiments, both types of input can be provided to the classifier 160. Accordingly, the regressor 156 can functionally obtain a broad range of information by virtue of the fact that it is trained on samples from multiple patients and multiple disease states.

One important aspect of the process 100 is that the regressor 156 is trained on peptide data that represents more than one disease state or condition. In other words, the process 100 does not train the regressor 156 only on data from a single condition and, thus, the classifier 160 is not limited to only identifying the single condition on which the machine learning system 150 was trained. Functionally, this means that the regressor 156 evaluates samples from as many patients and diseases as desired and, accordingly, generates an embedder that generally contains general knowledge about immune function and immune response to disease. The embedder can be used to generate the input provided to the classifier 160, which allows the classifier 160 to take advantage of the broad learning obtained from performing a regression on samples from many patients and with multiple diseases. As discussed in further detail below, by training the regressor 156 on data representing multiple disease states or conditions, the performance of the classifier 160 is improved in multiple respects. First, the classification performance of the classifier 160 is improved across the entire range of disease states or conditions on which the regressor 156 was trained. Second, the classifier 160 demonstrates an improved robustness to noise (e.g., Gaussian noise) in the peptide data. Third, the regressor 156 learns relationships between the various disease states or conditions that are applicable to additional disease states or conditions, which could in turn be used to improve the performance of the classifier 160 on new, unseen disease and thereby allows the classifier 160 to potentially be used to identify additional disease states or conditions on which the classifier was not trained.

In some embodiments, the classifier trained 104 as described above can subsequently be used to identify a disease state or condition exhibited by a new sample from a patient. In some embodiments, the classifier could be used to identify the presence of the disease states or conditions on which the classifier was trained. In other embodiments, the classifier could be used to identify the presence of the disease states or conditions on which the classifier was not trained. In some embodiments described further herein, a clustering algorithm (e.g., a Uniform Manifold Approximation and Projection (UMAP) algorithm, a Principal Component Analysis (PCA) algorithm, a hierarchical clustering algorithm, a k-means algorithm, an expectation-maximization algorithm, an HCS clustering algorithm, or the like) is applied to a set of weight and bias values of a trained electronic neural network to generate disease maps of populations.

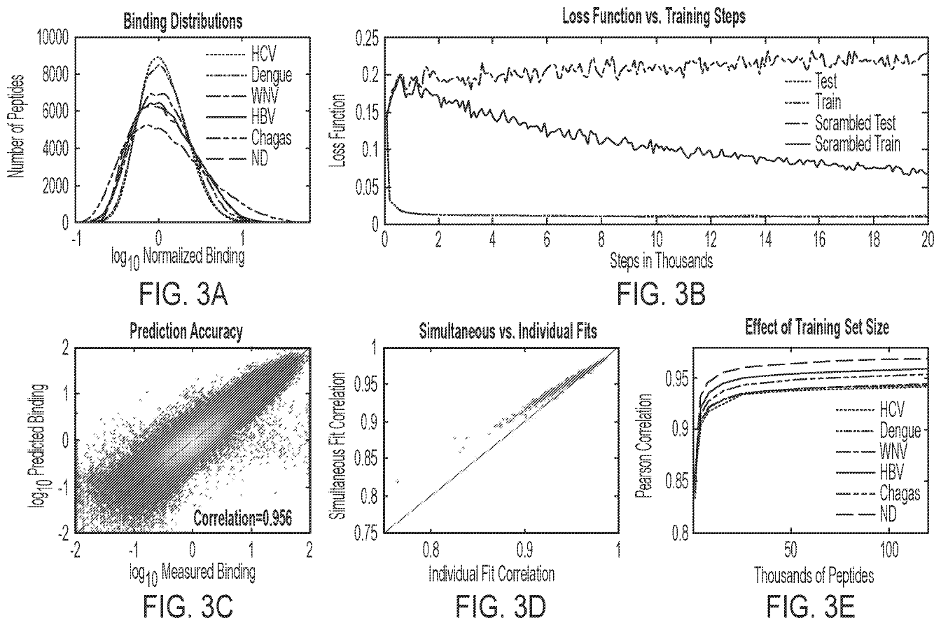

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of peptide sequence and binding value pair data sets suitable for use as a training corpus as disclosed herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. DESCRIPTION OF EXAMPLE EMBODIMENTS

### Example: Exploring the Sequence Space of Molecular Recognition Associated with the Humoral Immune Response

In the present example, the hypothesis that the amino acid sequence space of antigens or epitopes involved in an immune response is locally smooth relative to antibody binding was tested in a model system by synthesizing a very sparse and nearly random sample of short amino acid sequences (peptides around 10 residues in length) and incubating them with serum from 6 different sample cohorts (5 infectious disease cohorts and an uninfected cohort). The total IgG binding in each sample to each of these sequences was recorded, and a neural network was trained and used to create a quantitative relationship capable of predicting the IgG binding of each sample to any sequence. By combining these relationships together, one can effectively produce a quantitative description of the humoral immune response to each disease as a function of molecular recognition by any linear amino acid sequence in a similar context.

Common methods to generate antibody binding profiles associated with an immune response to infection generally focus narrowly on a particular pathogen, displaying short overlapping peptides presented on microarrays or in phage display libraries generated by tiling antigens or entire proteomes. However, this does not provide an unbiased view of the molecular recognition sequence space as it is strongly biased by focusing on previously identified antigens. Panning of phage or bacterial peptide display libraries coupled with next generation sequencing have provided broader binding profiles, but these are also biased; panning of such libraries focuses on enriched binders, limiting the descriptive information of low and non-binding sequences required for comprehensive quantitative modeling of immune response interactions.

Over the past decade, a number of studies have been published using high density peptide arrays as a tool for antibody binding profiling. A key feature of these arrays is that the peptide sequences are chosen to cover sequence space as evenly as possible, rather than focusing on biological sequences or known epitopes. This “immunosignature” approach captures mostly low to moderate affinity interactions with the array peptides and has been shown to enable robust differentiation of more than 30 different infectious and chronic diseases. The method involves applying a small amount of diluted sample of serum to a dense array of peptides with nearly random sequences of amino acids, typically with >100,000 distinct peptide sequences of about 10 amino acids in length. Binding of IgG or another circulating antibody isotype to the peptides on the array is then detected quantitatively using a fluorescently labeled secondary antibody and imaged by an array scanner. Based on the pattern of binding seen in case and control samples, statistical feature selection is performed, and classifier models can be built to distinguish one disease from another.

As noted herein, the peptide sequences used on the array were selected with the goal of sparsely covering all combinatorial sequence space as evenly as possible (within the constraints of the synthetic method used to make the array). However, with just ˜10⁵ sequences, only a tiny fraction of the >10¹³ total possible 10-mer amino acid sequences are available for defining the binding profiling on the array. As a result, it is highly unlikely that any sequences on the array correspond directly to a cognate linear epitope(s) in a pathogen proteome, much less a conformational (structural) epitope. In fact, for the arrays used in the published work noted above, only 16 of the natural 20 amino acids were used to synthesize the peptide library, further constraining the ability to directly represent arbitrary natural peptide sequences. Thus, the information used to differentiate diseases is contained in a molecular recognition profile of antibody binding to an extremely sparse sample of all possible sequences. What is surprising is how apparently differentiating this recognition information content is, despite its sparseness, as evidenced by its ability to discriminate disease accurately. This is consistent with the hypothesis presented above. One way in which the IgG binding to an extremely sparse sampling of nearly random sequence space could provide sufficient information to specifically distinguish the immune response of a disease is if the IgG binding features in linear sequence space were broad and smooth with respect to modest sequence changes, so that many different sequences can provide information about binding to a particular epitope.

The peptide arrays described above provide an appropriate model system for developing a comprehensive, quantitative relationship between amino acid sequences in a defined sequence space and the molecular recognition profile of an immune response. Machine learning algorithms have been commonly used to develop sequence-based models predicting binding of proteins to peptides, antibodies, and DNA. These studies describe the identification of anti-microbial peptides, infectious viral variants that escape protection, potential epitopes on target antigens, high antibody binding regions on target proteins, and optimization of target DNA sequences for Transcription factors (TFs). To do this, primarily two approaches have been used: 1) introducing single or multiple point mutations on a target site with known function to identify desired leads, and 2) use of proteomes of interest or known antigenic proteins to predict epitopes. As described above, the narrow nature of the dataset biases the output and thus limits the predictive capability of such algorithms. For example, epitope prediction tools such as BepiPred-2.0 are generally developed using known antigens derived from the crystal structures of antibody-antigen complexes, making them potentially biased towards high affinity interactions. Others have attempted to overcome this limitation by using an expanded molecular interaction modeling to cover a broader range of ligands by applying multivariate regression to serum antibody binding to a library of 255 random peptides. Using such an approach, serum antibody binding from naïve mice was well modeled by relating peptide composition to binding intensity, however binding of serum antibodies from previously infected mice was poorly modeled. This suggests that to successfully model disease specific affinity matured antibodies, a more complex library of peptide ligands and accounting of peptide sequence in the mathematical model are needed.

Recently, our group used an unbiased approach to develop sequence-based predictive models for the binding data of nine different, well-characterized isolated proteins to the peptide arrays described above. Binding patterns of each protein were recorded, and a simple feed-forward, back propagation neural network (NN) model was used to relate the amino acid sequences on the array to the binding values. Remarkably, it was possible to train the network with 90% of the sequence/binding value pairs and predict the binding of the remaining sequences with accuracy equivalent to the noise in the measurement (the Pearson correlation coefficients (R) between the observed and predicted binding values were equivalent to that between measured binding values of multiple technical replicates, and in some cases as high as R=0.99). In fact, accurate binding predictions (R>0.9) for some protein targets could be achieved by training on as little as a few hundred randomly chosen sequence/binding value pairs from the array. In addition, the binding predictions were specific; the neural networks captured not only the bulk binding of individual proteins but the differential binding between proteins. Finally, training on weakly binding sequences effectively predicted the binding values of the strongly binding sequences on the array with binding levels 1-2 orders of magnitude greater. The key point is that a very sparse sampling of total amino acid sequence space was sufficient to describe the entire combinatorial sequence space of peptide binding with high statistical accuracy.

These protein-array binding results again imply that the topology of sequence space associated with protein binding is broad and reasonably smooth, with one local binding feature in that space encompassing many sequences. In the work described above, the fact that a statistically accurate, binding model describing the ˜1012 possible sequences in the model sequence space sampled by the array (16 possible amino acids with each peptide about 10 residues in length) could be derived from binding to an array of 105 sequences, implies that the binding features alluded to above must consist of at least 107 sequences on average. Polyclonal serum antibody binding is clearly a much more complex and specific system than isolated proteins as it involves a large antibody repertoire including the dominant affinity matured antibodies. However, as mentioned above, the finding that the immunosignature approach can differentiate disease states suggests that the molecular recognition of the immune system in terms of specific disease response may also be describable by measuring the molecular recognition of a very sparse sampling of sequences out of the entire combinatorial binding space, as it is for isolated protein/peptide binding. If so, it should be possible to develop a comprehensive and quantitative relationship between an amino acid sequence in our model sequence space and binding associated with the specific immune response to a given disease.

Here, neural network-based models were used to build quantitative relationships for sequence-antibody binding using serum samples from several infectious diseases: a set of closely related flaviviridae viruses (Dengue Fever Virus, West Nile Virus and Hepatitis C Virus), a more distantly related hepadnaviridae virus (Hepatitis B Virus) and an extremely complex eukaryotic trypanosome (Chagas Disease, Trypanasoma cruzi). This allowed a thorough evaluation of the differential information content of the array information and the ability of the machine learning algorithms to accurately capture that information. The ability of the system to enhance disease differentiation by effectively combining peptide sequence information with binding information was also explored.

### Methods

### Peptide Arrays:

The peptide arrays used were produced locally at ASU, via photolithographically directed synthesis on silicon wafers using methods and instrumentation common in the electronics fabrication industry. The synthesized wafers were cut into microscope slide sized pieces, each slide containing a total of 24 peptide arrays. Each array contained 122.926 unique peptide sequences that were 7-12 amino acids long. A 3 amino acid linker consisting of GSG was attached to each peptide and connected the C-terminus to the array surface via amino silane. The peptides were synthesized using 16 of the 20 natural amino acids (A,D,E,F,G,H,K,L,N,P,Q,R,S,V,W,Y) in order to simplify the synthetic process (C and M were excluded due to complications with deprotection and disulfide bond formation and I and T were excluded due to the similarity with V and S and to decrease the overall synthetic complexity and the number of photolithographic steps required). The arrays were created in 64 photolithographic steps (4 rounds through addition of the 16 amino acids) and sequences were chosen from the set to cover all possible sequences as evenly as the synthesis would allow. The 64-step limitation was important to keep the number of mask alignments during photolithographic synthesis low enough to maintain high sequence fidelity. One loses some sequence possibilities with this approach (for example, there are serious constraints on sequences with 3 or more repeated amino acids), but because it is possible to select which ones are made on the array, one can still provide a fairly even coverage of the possible sequence space.

### Serum Samples:

Serum samples were collected from three different sources: 1) Creative Testing Solutions (CTS), Tempe, AZ 2) Sera care 3) Arizona State University (ASU) (Table 1). The dengue serotype 4 serum samples were collected from 2 of the above sources: 30 samples were purchased from CTS and 35 samples were purchased by Lawrence Livermore National Labs (LLNL) from Sera Care before they were donated to Center for Innovations in Medicine (CIM) in the Biodesign Institute at ASU. Uninfected/control samples consisted of 200 CTS samples and 18 samples from healthy volunteers at ASU. The rest of the infectious case samples were purchased from CTS. All case donors were reported as asymptomatic at the time of collecting serum. The Chagas disease serum samples were tested seropositive with a screening test (Abbott PRISM T. cruzi (Chagas) RR) based on the presence of T. cruzi specific antibodies and subsequently confirmed as T. cruzi seropositive using a confirmatory test. The confirmatory test was either a radioimmunoprecipitation (RIPA) or anti-T. cruzi enzyme immunoassay, Enzymatic Immuno Assay (EIA) (Ortho T. cruzi EIA). West Nile Virus (WNV) positive samples were identified at CTS by assaying for WNV RNA using a nucleic acid amplification (NAT) assay (Procleix® WNV Assay). The samples were also tested in an EIA (WNV Antibody (IgM/IgG) ELISA, Quest Diagnostics) to detect IgM and IgG antibodies. Samples with both antibody isotypes detected in the EIA were further tested in a reverse transcriptase-polymerase chain reaction (RT-PCR) based assay. HBV samples were screened (ABBOTT PRISM HBsAg Assay Kit) for the detection of HbsAg and NATAbbott PRISM HBC RR, reactive samples were confirmed non-reactive for HCV and HIV RNA in a NAT (PROCLEIX ULTRIO ELITE ASSAY) and reactive in an HBV NAT assay, and finally considered as HBV positive using a HBsAg Neutralization assay. If samples tested negative for nucleic acids, then they were tested for anti-HBC antibodies (Abbott PRISM HBC RR). In the case of HCV, a test approach similar to HBV was used with an additional test, a highly anti-HCV specific assay (recombinant immunoblot assay, RIBA) to confirm the samples as HCV positive. For uninfected controls, samples were tested as non-reactive in a NAT assay and hence confirmed as uninfected or healthy. Dengue serotype 4 samples were assayed for anti-NS1 IgG as Dengue positive, and the serotype was confirmed by an indirect immunofluorescence test. Serum samples were frozen at the time of collection and not thawed before received as aliquots in CIM.

### Sample Processing and Serum IgG Binding Measurement:

Serum from the 6 sample cohorts (5 disease cohorts and uninfected) were diluted (1:1) in glycerol and stored at −20° C. Before incubation, 2 μl of each serum sample (1:1 in glycerol) was prepared as 1:625 dilution in 625 μl incubation buffer (phosphate buffered saline with 0.05 Tween 20, pH 7.2). The slides, each containing 24 separate peptide arrays were loaded into an ArrayIt microarray cassette (ArrayIt, San Mateo, CA). Then, 20 μl of the diluted serum (1:625) was added on a Whatman 903T Protein Saver Card. From the center (12 mm circle) of the protein card, a 6 mm circle was punched, and put on the top of each well in the cassette, and covered with an adhesive plate seal (3M, catalogue number: 55003076). Incubation of the diluted serum samples on the arrays were performed for 90 minutes at 37° C. with rotation at 6 rpm in an Agilent Rotary incubator. Then, the arrays were washed 3 times in distilled water and dried under nitrogen. A goat anti-human IgG(H+L) secondary antibody conjugated with either AlexaFluor 555 (Life Technol.) or AlexaFluor 647 (Life Technol.) was prepared in 1×PBST pH 7.2 to a final concentration of 4 nM. Following incubation with primary antibodies, secondary antibodies were added to the array, sealed with a 3M cover and incubated at 37° C. for 1 hour. Then the slides were washed 3 times with PBST (137 mM NaCl. 2.7 mM KCl, 10 mM Na₂HPO₄, and 1.8 mM KH₂PO₄. 0.1% Tween (w/v)), followed by distilled water, removed from the cassette, sprayed with isopropanol and centrifuged dry dried under nitrogen, and scanned at 0.5 μm resolution in an Innopsys (Chicago, Ill.) Innoscan laser scanner, excitation 547 nm, emission 590 nm. For extraction of fluorescence intensities for each feature on the array representing a unique peptide sequence, images in 16-bit TIFF format were aligned to a grid containing the identifiers and sequences for each peptide using GenePix Pro 6.0 (Molecular Devices, San Jose, CA). The raw fluorescence intensity data were provided as a tab delimited text file in the GenePix Results (‘gpr’) file format.

### Binding Analysis Using Neural Networks:

The neural network used to relate peptide sequence on the array to the measured binding of total serum IgG is very similar to that described previously for relating sequence to protein binding on peptide arrays. The amino acid sequences were input as one-hot representations. An encoder layer linearly transforms each amino acid into a real-valued vector. The encoder matrix values were optimized during the training. The encoder vectors for each amino acid in the sequence were then concatenated together in the same order as the sequence. A feed-forward, back-propagation neural network was then trained on a fraction of the peptide sequence/binding value pairs and the resulting model was used to predict the binding value of the remaining peptide sequences not involved in the training (the test set). An L2 loss function (sum of squared error) was used for the training. The model performance was assessed by calculating the Pearson correlation coefficient between the measured and predicted binding values in the test dataset. Unless otherwise stated, the neural networks used in this work were trained on all samples simultaneously (the output layer and target matrix each consisted of a number of columns equal to the number of different samples, so for every sequence input, one value was predicted for each sample).

The neural network was trained using binding values from the peptide array that were normalized by the median binding value of all peptides in that sample. The log₁₀ of the normalized values were then used in subsequent analyses (any zeros in the dataset were replaced by 0.01× the median prior to taking the log). Pearson correlation coefficients (R) reported for predicted vs. measured binding values were based on the log₁₀ data and represented the average of multiple random selections of training and test peptide sequences.

### Results

### Study Design and Initial Analysis:

The serum samples shown in Table 1 were incubated on identical peptide microarrays as described in Methods and bound IgG was detected via subsequent incubation with a secondary anti-IgG antibody. The peptide sequence ‘QPGGFVDVALSG’ is present on the array as a set of replicate features (n=276). This peptide sequence gives a consistently moderate to strong binding value from sample to sample and is used to assess the intra-array spatial uniformity of antibody binding intensities. Poor quality arrays were defined as having an intra-array replicate feature coefficient of variation (CV)>=0.3 for this peptide sequence. In addition, some arrays showed significant physical defects or overall increases in binding intensity between different regions of the array (collectively these are referred to as “High CV samples”). In all, 20% of the 679 arrays measured were excluded from the initial part of the analysis but considered in the last section which focuses on using sequence data to remove noise from the arrays. Thus, 542 arrays total were considered “Low CV Samples” in Table 1.

TABLE 1

| DISEASE | SAMPLE | SAMPLES | SAMPLES | GENOME |
|---|---|---|---|---|
| COHORT | SOURCE | COLLECTED | USED | SIZE |
| Dengue Virus, | CTS and | 65 | 57 | 9600 |
| Serotype 4 | Sera Care | | | |
| (DENV4) | | | | |
| West Nile Virus | CTS | 100 | 74 | 11,000 |
| (WNV) | | | | |
| Hepatitis C Virus | CTS | 100 | 77 | 11,000 |
| (HCV) | | | | |
| Hepatitis B Virus | CTS | 100 | 86 | 3200 |
| (HBV) | | | | |
| Chagas | CTS | 100 | 70 | 105M |
| Uninfected or | CTS and | 218 | 177 | — |
| control | ASU | | | |

### Comparison of Average Binding Profiles of Peptides to Serum IgG.

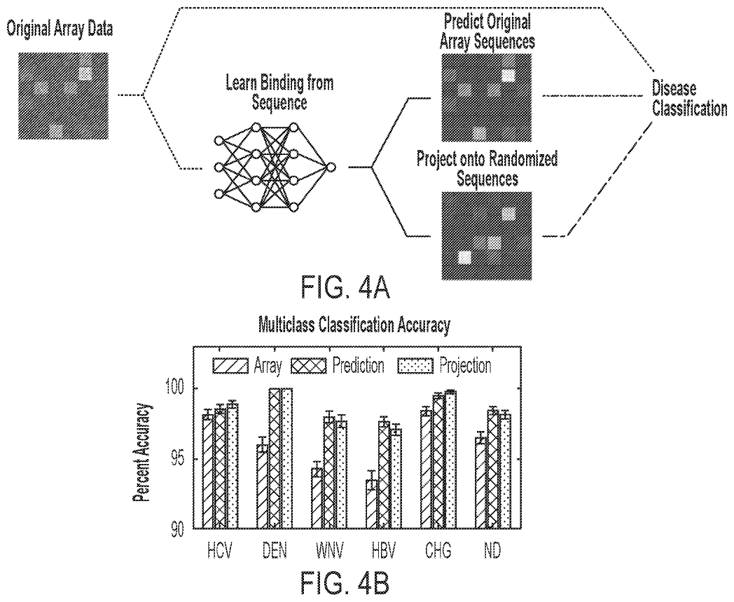

FIG. 3(A) shows the cohort average serum IgG binding intensity distributions of the 122,926 unique peptide sequences. The samples were all median normalized prior to averaging each peptide binding value within the cohort. The log₁₀ of the average binding is displayed on the x-axis as the log distributions are much closer to a normal distribution than are the linear binding values. The three Flaviviridae viruses (HCV, Dengue and WNV) have sharper distributions (smaller full width at half maximum) than the other samples, while Hepatitis B shows a distribution width similar to uninfected donors. Chagas disease has a broader binding distribution than the others, with a long tail on the high binding side. It seems reasonable that small proteome viruses result in a more focused immune response, while larger proteomes give rise to broader binding profiles. What is less intuitive is that for the small viruses some of the higher binding antibodies are lost. However, it is important to remember that the array peptides have no relationship to the viral proteomes or indeed any biological proteome, except by chance. Thus, what is lost in terms of array binding in uninfected samples, may well be gained in more specific binding not immediately apparent.

### Neural Network Analysis:

A fundamental hypothesis of this study is that it should be possible to accurately predict the sequence dependence of antibody binding, both in terms of accurately representing the IgG binding to each peptide sequence in individual serum samples and in terms of the ability of the neural network to capture sequence dependent differences in IgG binding between samples and between cohorts. Towards this end, samples were analyzed using feed forward, back propagating neural network models in two different ways. In one approach, each sample was analyzed separately so that a neural network model was developed for every serum sample independently (the loss function depended only on a single sample). In the second approach, all samples were fit together with a single neural network such that the 542 different low CV sets of binding values were included in the same loss function. In both cases, the optimized network involved an input layer with an encoder matrix (see Methods), two hidden layers with 350 nodes each and an output layer whose width corresponded to the number of target samples (1 for individual fits and 542 when all samples were fit simultaneously). The loss function used was the sum of least squares error based on the comparison of the predicted and measured values for the peptides in the sample.

### The Neural Network Uses the Sequence Information to Rapidly Converge on a Solution.

FIG. 3B shows the rate at which the loss function drops during training using the simultaneous fitting approach in which all samples are analyzed together. When the correct sequence is paired with its corresponding binding value (bottom two lines, FIG. 3B), the value of the loss function drops rapidly and the values for the training set and test set drop in concert; there is almost no overfitting.

As a control, the same neural network was used to analyze data in which the order of the peptide sequences was randomized relative to their binding intensities. One would not expect any relationship between sequence and binding under these circumstances. In this case, the loss function value for both the training and test initially rise slightly followed by a slow drop for the training set of peptides over the entire training period and a slow rise for the test set (top most trace: test, second to top most trace: train). This implies that the neural network is rapidly converging on a true relationship between the sequences and their binding values. However, in the absence of such a relationship, the neural network slowly overfits based on noise and the representation of an independent test set becomes increasingly worse.

### The Neural Network Results in a Comprehensive Binding Model Applicable Across Combinatorial Sequence Space.

FIG. 3C shows a scatter plot comparing the predicted and measured values from a neural network model fitting all the samples simultaneously. In this case, the model was trained on 95% of the peptide sequence-binding pairs for each sample, randomly selected (but the sample peptide sequences for all samples), with the remaining 5% or 6,146 peptide sequences excluded from training and used for model testing (that is 6,146 binding values for each of the 542 low CV samples=˜3.3 million binding values in the training set). Only the test set values are displayed in FIG. 3C. Since the sequences used on the array are nearly random, these sequences should be statistically equivalent to any randomly selected set of sequences from the combinatorial space of possible sequences sampled by the array (peptides of about 10 residues utilizing any of 16 amino acids give rise to a combinatorial sequence space of ˜10¹² possible sequences). Thus, the model provides a comprehensive and statistically accurate means of predicting the binding of any random sample of sequences in this sequence space. (This does not mean the binding prediction never fails, only that for any random sample of sequences, it gives statistically high accuracy.) The Pearson correlation coefficient (R) between the measured and predicted values for the test sequences shown is 0.956. Repeating the training 100 times with randomly selected train and test sets gives an average 0.956 with a standard deviation of 0.002. The correlation coefficient between measured and predicted binding for the 95% of the sequences used to training the neural network was 0.963+/−0.002. Thus, there is almost no overfitting associated with the model (the quality of fit between the test and train data is similar). Some cohorts and some samples were better represented than others, but for the vast majority of the samples, the correlation coefficients are greater than 0.9.

There are Commonalities in the Binding of Each Sample that Make Simultaneous Modeling of all Samples More Accurate than Individual Neural Network Models.

As stated above, it is possible to either build entirely independent neural network models for each of the samples considered or to build models that fit all of the samples simultaneously. FIG. 3D shows a direct comparison of the measured vs. binding correlation coefficient of each sample using the simultaneous and individual model approaches. In almost every case, the simultaneous model is more accurate, providing a small, but significant, improvement in correlation coefficient. This implies that the network can more accurately learn the commonalities between IgG binding from serum when all samples are fit at once. In the simultaneous model, these common features will be incorporated into the 2 hidden layers of the neural network and the differences between samples will be incorporated into the output layer (the final weight matrix), with a separate column in that layer giving rise to the binding values for each sample. Simultaneous modeling of all the samples was used for the remainder of the analyses.

10³ to 10⁴ Peptides are Enough to Provide a Reasonable Description of the Entire Combinatorial Peptide Sequence Space.

Neural network models were trained with different numbers of randomly selected peptides and binding was predicted for the remaining portion of the peptides. FIG. 3E explores the dependence of the overall correlation coefficient between measured and predicted binding values for the test set of each of the sample cohorts as a function of the number of peptides used in the training. When at least 10,000 peptide sequences are used to train the neural network, the correlation coefficient is >0.9 for all cohorts, and the correlation is >0.85 when the model is trained using only 2,000 peptides. This implies that the binding features in this model sequence space are very broad, encompassing many millions of sequences so that even a very sparse sampling of this ˜10¹² sequence combinatorial space provides a reasonably high-resolution map of its topology. The correlation coefficients do continue to increase slowly throughout the increase in the size of the training set. Thus, even though a relatively small set of peptides gives a reasonable overall picture, the predictive power of the relationship continues to improve with more data, and if even more peptide sequences were available for training than the entire 122,926 peptides on the array an improved prediction would be expected.

### The Neural Network Learns Distinguishing Characteristics of Cohorts:

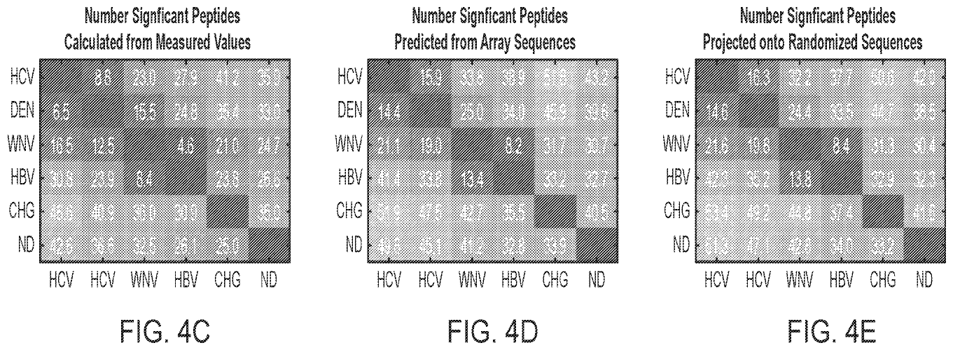

Note that for this section of the analysis, 100 ND samples were selected out of the 177 and used in order to better balance the numbers of samples in each cohort. FIG. 4A is a schematic of three approaches to disease classification and discrimination. The small equally sized dashed line is the simple statistical pathway (immunosignaturing). Here the binding values are considered simply as a vector of values (no sequence information is used). These can be either fed into a classifier (FIG. 4B) or used to determine number of significant peptides that distinguish diseases (FIG. 4C), as described below. Alternatively, the neural network can be used to determine a sequence/binding relationship. This relationship can either be used to recalculate predicted binding values for the array peptide sequences, forcing the data to always be consistent with the sequence (dashed line with pairs of equally sized small intervening dots), or it can be projected onto a completely new set of sequences (in in silico array, dashed line with single smaller sized intervening dashes), and those projected binding values used in classification or determining the number of significant distinguishing peptides between disease pairs.

### Values Predicted by the Neural Network Result in Better Classification of Disease.

FIG. 4B shows the result of applying a multiclass classifier, either to the measured binding values, the binding values predicted for the array sequences or binding values predicted for in silico generated sequences. A simple classifier was built using a neural network with a single hidden layer and 300 nodes. Peptide features were chosen using a test between each cohort and all others. Either 20 features (the measured data) or 40 features (the two predicted data sets) were used per cohort, with the number of features chosen to be optimal for the dataset. The training target is a one-hot representation of the sample cohort identity, and the network is set up as a regression. One fifth of the samples were randomly selected for each repetition of the classification and the process was repeated 100 times. Each test sample was then labeled as the cohort with the largest value in the resulting output vector. For every cohort, with the possible exception of HCV, classification was improved relative to measured array values (bars with single diagonal lines) when using the predicted values. This was true whether using predicted values for the array sequences (bars with cross-hatched lines) or values resulting from projection of the trained network on randomized in silico array sequences (bars with dots).

The Predicted Values of the IgG Binding to Array Sequences Distinguish Cohorts Better than the Measured Values, Even Predicted Values for a Set of Randomized Sequences.

FIGS. 4C-E show another approach to measuring the ability to distinguish between cohorts. The number of peptide binding values that are significantly greater in one cohort (on the Y-axis) compared to another (on the X-axis) are shown in each grid. Significance was determined by calculating p-values for each peptide in each comparison using a T-test between cohorts. Significant peptides are those in which the p-value is less than 1/N (N=122,926) with a >95% confidence. FIG. 4A shows comparisons between cohorts using the measured data from the arrays. As one might expect, the sera from donors infected with the flaviviruses are most similar to one another in terms of numbers of distinguishing peptides. In general, they are more strongly distinguished from HBV (except for West Nile Virus) and very strongly distinguished from Chagas donors. If one follows, for example, the top row of FIG. 4A for HCV, moving to the right one sees that the numbers increase as more and more genetically dissimilar comparisons are made. West Nile virus is an exception in this regard. While it is more similar to the other flaviviruses than it is to Chagas, it is most similar, in terms of numbers of distinguishing peptides, to HBV (FIG. 4A).

FIG. 4B is the same as FIG. 4A except that in this case, the predicted values from the neural network model are used for the array sequences instead of the measured values. Because the network requires that a common relationship between sequence and binding be maintained for all sequences, it increases the signal to noise ratio in the system such that significantly more distinguishing peptides are identified in every comparison. The neural network was run 10 times and the results averaged.

FIG. 4C shows results in the same format as the other two panels, but this is uses the in silico generated sequences and projected binding values. These sequences were produced by taking the amino acids at each residue position in the original sequences and randomizing which peptide they were assigned to. This created an in silico array with a completely new set of sequences that had the same number, overall amino acid composition and average length as the sequences on the array to ensure a consistent comparison. The binding values for each sample were then predicted for this in silico array and those values were used in the cohort comparisons. The number of significant peptides identified using the new sequence set are identical to within the error for each comparison with the predictions from the actual array peptide sequences used in the training. Note that the result of generating ten different randomized in silico arrays was averaged.

### Understanding the Noise Reduction Properties of the Neural Network Modeling:

The results presented above show that by using the sequence/binding information to first train a neural network model and then predicting the binding using that model (on the same or a different set of sequences), it is possible to improve the signal to noise ratio in the data, at least in terms of differentiating between disease cohorts. To understand this in more detail, the effects of added noise on the data is explored.

### Gaussian Noise is Effectively Removed by the Model.

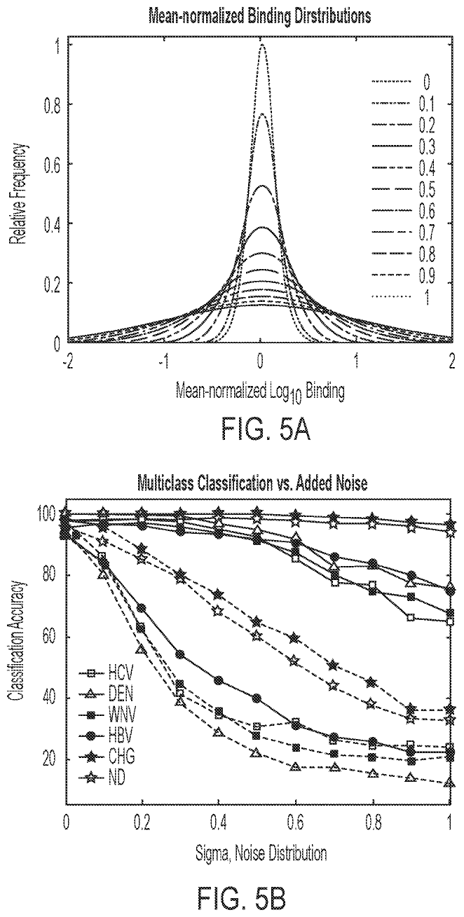

In FIG. 5, noise was artificially added to each point in the measured dataset by using a random number generator based on a gaussian distribution that was centered at the measured value:

$$f ( x ) = \frac{1}{σ \sqrt{2 π}} {e}^{- \frac{1}{2} {( \frac{x - μ}{σ} )}^{2}}$$

In the above equation, the mu (μ) is the log₁₀ measured binding value. Sigma (σ) was then varied from 0 to 1 to give different levels of added noise. Note that sigma=1 results in addition of noise on the order of 10-fold greater or less than the linear binding value measured (due to the log₁₀ scaling). FIG. 5A shows the resulting distribution of peptide binding values after adding noise. The peptide binding values were mean normalized across all cohorts and then plotted as a distribution, for each cohort (since this is the log₁₀ of the mean normalized value, the distributions are centered at 0). As sigma is increased, the width of the resulting distribution after adding noise increases dramatically.

FIG. 5B plots the multi-class classification accuracy of each dataset for each sample cohort as a function of sigma (this uses the same classifier as FIG. 4). The classification accuracy of the original measured data with increasing amounts of noise added drops rapidly (dashed lines). Since this is a 6-cohort multi-class classifier, random data would give an average accuracy of ˜17%. The measured values with added noise approach that accuracy level at the highest noise. However, by running the data through the neural network and then using predicted values for the same sequences as are on the array, the accuracy changes only slightly for sigma values up to about 0.5 and then drops gradually with increased noise, but always well above what would be expected for random noise. Note that a sigma of 0.5 corresponds to causing the measured values to randomly vary between about 30% and 300% of their original values.

### Neural Network Predictions of Array Signals Improved Classification of High CV Samples.

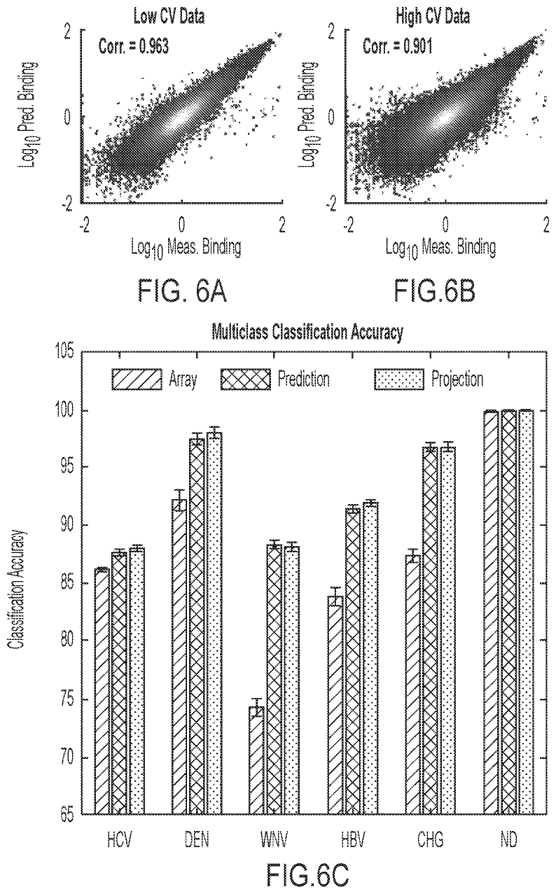

As described above, 137 samples were not used in the analyses above because they either had high CV values calculated from repeated reference sequences across the array or because there were visual artifacts such as scratches or strong overall intensity gradients across the array. A neural network model was applied to all of the 679 (542 low CV+137 high CV) samples simultaneously. Note that the model does not include any information about what cohort each sample belongs to, so modeling does not introduce a cohort bias. The overall predicted vs. measured scatter plots and correlations are given in FIGS. 6A and B for both the low CV data and for the high CV data (the number of points displayed was randomly selected by constant between datasets to make the plots comparable). Prediction of the binding values of the high CV data results in more scatter relative to measured values, due to the issues with those particular arrays.

In FIG. 6B, the measured and predicted values for the 542 low CV samples were used to train a multiclass classifier which was then used to predict the cohort class of the high CV samples. Three different data sources were used: 1) the measured array data (bars with single diagonal lines), 2) predicted binding values for the array peptide sequences based on the neural network model (bars with cross-hatched lines) and 3) projected values for a randomized set of sequences with the same overall size, composition and length distribution as the sequences on the array (bars with dots). The classifier used was the same as that in FIG. 4 and the number of features selected was optimized for the data source (20 features per cohort for the measured array data and 40 features per cohort for the two datasets based on the neural network predictions). In each case except for the non-disease samples, the use of predicted values resulted in a significantly better classification outcome.

### Discussion

### A Quantitative Relationship Between Peptide Sequences and Serum IgG Binding:

The work described above shows that it is possible to use a relatively simple neural network model to generate a comprehensive relationship between amino acid sequence and binding over a large amino acid sequence space using only a very sparse sampling of binding to that sequence space. As seen previously for isolated protein binding to sequences on these arrays, knowing the binding values of 10⁵ sequences allows one to predict, with high statistical accuracy, the values of any random subset of sequences among the ˜10¹² possible sequences. Indeed, a reasonably accurate prediction can be obtained with only thousands of sequences (FIG. 3F). As suggested previously for isolated proteins, this implies that the topology of the multidimensional binding vs. sequence space is locally broad and smooth such that more than ten million different sequences contain overlapping binding information relative to an immune reaction, at least for the model sequence space used in this analysis.

Clearly the model system used here to explore the relationship between antibody molecular recognition profiles and amino acid sequences has limitations. Only 16 of the 20 natural amino acids were used in this model for technical reasons. The sequences are also bound at one end to an array surface, and the other end has a free amine rather than a bond as would be seen in a protein. In addition, the array peptides are short, linear and largely unstructured. All of this limits the range of molecular recognition interactions that can be observed, and thus the level of generality of the conclusions, but this suggests that comprehensive and accurate structure/binding relationships for humoral immune responses should be possible to generate given binding data in a broader sequence context. Such relationships would be invaluable for epitope prediction, autoimmune target characterization, vaccine development, effects of therapeutics on immune responses, etc. Even this rather simple model system for sequence space already shows the ability to capture differential binding information between multiple diseases simultaneously, including diseases that involve closely related pathogens (FIG. 4).

The fact that one can develop comprehensive sequence/binding relationships within this model sequence space also explains, at least in part, why the immunosignature technology works as well as it does. Immunosignaturing technology as applied to diagnostics uses the quantitative profile of IgG binding to a chemically diverse set of peptides in an array followed by a statistical analysis and classification of the resulting binding pattern to distinguish between diseases. The approach has been successfully used to discriminate between serum samples from many different diseases and has been particularly effective with infectious disease, as exemplified by the robust ability to classify the diseases studied here (FIG. 4D). This raises the question, why would antibodies that are generated by the immune system to bind tightly and specifically with pathogens show any specificity of interaction to nearly random peptide sequences on an array? The success of the neural network in comprehensive modeling of the sequence/binding interaction provides an answer. The information about disease specific IgG binding is dispersed broadly in peptide sequence space, even in the interaction with sequences that themselves bind weakly and with low specificity, rather than being focused only on a few epitope sequences. It is not necessary to measure binding to the epitope if you have a selection of sequences that are broadly located in the vicinity of the epitope in sequence space.

### The Advantage of Analyzing Many Samples Simultaneously:

The results of FIG. 3E demonstrate that simultaneous neural network analysis of all samples from all cohorts provides a slightly more accurate overall description of binding than does sample by sample analysis. Conceptually, this suggests that there is enough in common between the antibody molecular recognition profiles of the various samples that using the same hidden layers to describe all of them, followed by an output layer with a distinct column describing each sample, is sufficient to both describe the general and specific binding interactions. Practically, it greatly reduces computation time; analyzing sample by sample on an 18-core machine with parallelization optimized required about a day, while simultaneous analysis took place in about 10 minutes (for the simple neural networks used here, implementation of GPUs does not substantially accelerate training).

### Using the Sequence/Binding Relationship to Eliminate Noise:

To evaluate the differential binding information learned by the neural network models, the numbers of distinguishing peptides per comparison between cohorts were compared between measured binding values on the array and the binding values predicted by the neural network. In FIG. 4, both the number of distinguishing peptides and the classification accuracy improved when the measured values for each array sequence were replaced by the corresponding predicted values. Effectively, the neural network focuses information from the entire peptide dataset on each of the predicted values. This has an averaging effect that is extremely potent. In FIG. 5, random noise (sequence independent variation) is purposely added to the array. Since the noise is added to the log₁₀ of the binding value, a sigma of 0.5 corresponds to a several-fold increase in the noise distribution width, as can be seen in FIG. 5A, and a sigma of 1 broadens the distribution of linear values by nearly an order of magnitude. As a result, multi-class classification performs poorly (FIG. 5B, dashed lines). However, because the neural network predictions effectively average the combined information from nearly 123,000 sequence/binding values in the generation of the sequence/binding relationship, random noise is dramatically reduced and a sigma of 0.5 has very little effect on classification and even a sigma of 1 provides reasonable results (FIG. 5, solid lines) given that this is a 6-cohort multi-class classification problem. This concept is taken further in FIG. 6, where arrays that for technical reasons were rejected because of excessive noise or physical artifacts affecting part of the array are included in the simultaneous analysis of all samples and their excess noise and defects are effectively repaired by comparison to other samples in the system. This is done without the network having any information about which cohort is which in the analysis. The implication for array based diagnostic applications is that replacing a purely statistical approach like immunosignaturing with a structure-based approach provides a means of eliminating noise that is unrelated to the binding properties of the sequences (obviously, the real patient to patient variance is not removed as these differences are based on proper binding of antibodies to specific sequences). More generally, structure-based analysis of large molecular arrays has the potential to overcome noise due to technical variation without the need of performing large numbers of replicates.

### Analysis Independent of the Specific Array Sequences Used:

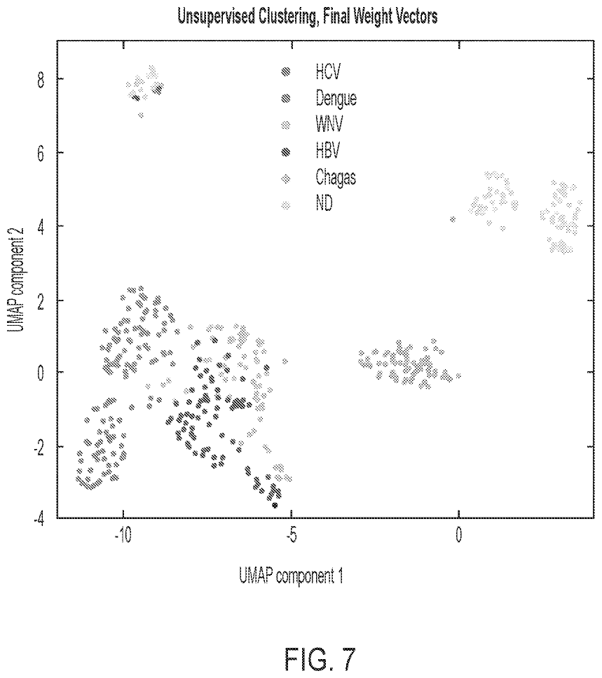

While particular sequences are used to train the neural networks, the networks themselves allow one to predict binding values for any sequence. As shown in both FIGS. 4 and 6, predicted values for a set of peptide sequences that approximately cover the same model sequence space as the array sequences discriminate between cohorts of samples just as well as predicted values of the original array sequences. In fact, it is the sequence/binding relationship that contains the discriminating information, and it is not necessary to use predicted binding to real sequences at all. For example, one can replace the predicted binding values of each sample with the columns in the final weight matrix plus bias of the trained neural network (there is one column in the final weight matrix generated for each sample which, in concert with the bias value, translates the output of the hidden layers into the specific binding values for that sample). This effectively replaces the ˜123,000 sequence/binding values with only a few hundred values (dictated by the number of nodes in the last hidden layer of the neural network). FIG. 7 shows an unsupervised clustering using the algorithm UMAP which reduced the 351 values of the final weight matrix for each sample plus the bias value to 2 components. The component values for each sample are plotted and the samples are color coded. The plot makes biological sense; the viruses are clustered together but fairly well separated into subgroups; chagas and uninfected donor samples are distantly separated. As was seen in FIG. 4, WNV and HBV are the hardest to distinguish, but the rest are almost completely distinguishable. Interestingly, there is one small cluster consisting of different kinds of samples completely separated from the others (upper left, FIG. 7). UMAP is a nonlinear clustering algorithm which looks for the most similar features in samples to determine clustering. Apparently, this cluster of individuals had some other unknown immunological stimulus in common that distinguished them from all others. The ability to detect such clusters could prove useful in bio-surveillance applications. FIG. 7 demonstrates that the cohort distinguishing information is contained in the 351 values of final weight matrix and bias; there is actually no need to use predicted binding values at all.

Note also that by working with sequence/binding relationships, rather than purely statistical comparisons of binding values for specific sequences, one can combine information from arrays that contain different peptides. As shown in FIG. 3F, when 50% of the array is used to predict the other 50%, the correlation coefficient on average is well over 0.9.

### The Topology of the Sequence/Binding Surface:

As pointed out in the introduction, antibody generation in B cells in response to infection starts with a very sparse sampling of large set of possible antibody sequence variants and is followed by a maturation process that occurs through rounds of genetic changes in B cells followed by antigen-stimulated proliferation. During maturation, apparently 4-6 amino acid changes out of about 30 amino acids involved in the compliment determining regions of the antibody are typical. This suggests that any B cell must optimize within a region of multidimensional sequence space that includes about 5×10¹¹ sequences during the course of the maturation process (20⁵×the number of ways of picking 5 amino acids from 30). This type of sparse sampling and gradient ascent optimization only works if two conditions are met with regard to the multidimensional binding surface encompassing antibody sequence space. First, for such sparse sampling to work at all, there must be a broad set of related antibody sequences that bind the antigen to some extent and includes the mature antibody sequence. Narrow topological features in the multidimensional sequence/binding space would be missed entirely by sparse sampling. Second, for a gradient ascent approach for maturation to work, these features must be locally smooth; it must be possible to climb the hill via many different paths and end up at or near the same binding capability.

The current study explores the inverse situation. Rather than sparse sampling of the antibody sequence space probing the topology of that binding surface, here sparse sampling of target sequence space was performed. However, one might expect the two to mirror one another. The fact that a neural network can learn to predict antibody binding accurately and comprehensively across sequence space using binding from a sparse sample of possible sequences says both that the regions of sequence space capable of binding to the IgG produced in response to disease are very broad and that the relationship between sequence and binding is well-behaved mathematically (infrequent discontinuities and relatively smooth surfaces across each functional feature). FIG. 3F shows that training the neural network on even a few thousand peptide sequence/binding pairs allows it to predict binding values for sequences not used in the training with reasonable accuracy, implying that the size of the features in sequence/binding space encompass at least tens of millions of different sequences (there are ˜10¹² total sequences possible in the sequence space explored here but only ˜10⁴ are needed for a solid prediction). In fact, the multidimensional features in sequence/binding space are probably much broader since there likely need to be many sequences sampled on each feature to create a predictive model. If the features were not locally smooth, accurate interpolation between measured sequences would not be possible; one would not be able to predict the binding characteristics of sequences not present in the original training set.

While it is true that the model sequence/binding space explored here is limited, as described above, the comparison to B cell maturation supports the idea that the concept is general, and it should be possible to create accurate sequence/binding relationships for essentially any humoral immune response given a modest sampling of appropriate sequence-context binding data. The practical implications of that are significant.

Some further aspects are defined in the following clauses:
Clause 1: A computer-implemented method of generating a disease map of a population, the method comprising applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.
Clause 2: The computer-implemented method of Clause 1, wherein at least one of the disease states is known.
Clause 3: The computer-implemented method of Clause 1 or Clause 2, wherein at least one of the disease states is unknown.
Clause 4: The computer-implemented method of any one of the preceding Clauses 1-3, wherein at least one of the disease states comprises an infectious disease state.
Clause 5: The computer-implemented method of any one of the preceding Clauses 1-4, wherein the disease map comprises clusters of the disease states represented in a two or more dimensional space.
Clause 6: The computer-implemented method of any one of the preceding Clauses 1-5, wherein the clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and/or an HCS clustering algorithm.
Clause 7: The computer-implemented method of any one of the preceding Clauses 1-6, wherein the set of weight and bias values is a final set of weight and bias values of the trained electronic neural network.
Clause 8: The computer-implemented method of any one of the preceding Clauses 1-7, further comprising producing the peptide sequence and binding value pair data sets from samples obtained from the reference subjects in the population.
Clause 9: The computer-implemented method of any one of the preceding Clauses 1-8, further comprising determining whether a test subject has at least one of the disease states using a peptide sequence and binding value pair data set obtained from the test subject and the trained electronic neural network and/or the disease map.
Clause 10: The computer-implemented method of any one of the preceding Clauses 1-9, further comprising generating at least one therapy recommendation for the test subject based at least in part on a determination that the test subject has the at least one of the disease states.
Clause 11: The computer-implemented method of any one of the preceding Clauses 1-10, further comprising administering one or more therapies to the test subject based at least in part on the therapy recommendation for the test subject.
Clause 12: The computer-implemented method of any one of the preceding Clauses 1-11, further comprising generating at least one iteration of the disease map at a time point that differs from a time point at which the disease map was generated.
Clause 13: The computer-implemented method of any one of the preceding Clauses 1-12, further comprising monitoring an immune status measure of the population and/or an occurrence of a known disease state or an unknown disease state in the population using the disease map and the iteration of the disease map.
Clause 14: The disease map produced by the method of any one of the preceding Clauses 1-13.
Clause 15: A system for generating a disease map of a population using an electronic neural network, the system comprising: a processor; and a memory communicatively coupled to the processor, the memory storing instructions which, when executed on the processor, perform operations comprising: applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.
Clause 16: The system of Clause 15, wherein at least one of the disease states is known.
Clause 17: The system of Clause 15 or Clause 16, wherein at least one of the disease states is unknown.
Clause 18: The system of any one of the preceding Clauses 15-17, wherein at least one of the disease states comprises an infectious disease state.
Clause 19: The system of any one of the preceding Clauses 15-18, wherein the disease map comprises clusters of the disease states represented in a two or more dimensional space.
Clause 20: The system of any one of the preceding Clauses 15-19, wherein the clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and/or an HCS clustering algorithm.
Clause 21: The system of any one of the preceding Clauses 15-20, wherein the set of weight and bias values is a final set of weight and bias values of the trained electronic neural network.
Clause 22: The system of any one of the preceding Clauses 15-21, wherein the instructions which, when executed on the processor, further perform operations comprising: determining whether a test subject has at least one of the disease states using a peptide sequence and binding value pair data set obtained from the test subject and the trained electronic neural network and/or the disease map.
Clause 23: The system of any one of the preceding Clauses 15-22, wherein the instructions which, when executed on the processor, further perform operations comprising: generating at least one therapy recommendation for the test subject based at least in part a determination that the test subject has the at least one of the disease states.
Clause 24: The system of any one of the preceding Clauses 15-23, wherein the instructions which, when executed on the processor, further perform operations comprising: generating at least one iteration of the disease map at a time point that differs from a time point at which the disease map was generated.
Clause 25: The system of any one of the preceding Clauses 15-24, wherein the instructions which, when executed on the processor, further perform operations comprising: monitoring an immune status measure of the population and/or an occurrence of a known disease state or an unknown disease state in the population using the disease map and the iteration of the disease map.
Clause 26: A computer readable media comprising non-transitory computer executable instructions which, when executed by at least one electronic processor, perform at least: applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.
Clause 27: The computer readable media of Clause 26, wherein at least one of the disease states is known.
Clause 28: The computer readable media of Clause 26 or Clause 27, wherein at least one of the disease states is unknown.
Clause 29: The computer readable media of any one of the preceding Clauses 26-28, wherein at least one of the disease states comprises an infectious disease state.
Clause 30: The computer readable media of any one of the preceding Clauses 26-29, wherein the disease map comprises clusters of the disease states represented in a two or more dimensional space.
Clause 31: The computer readable media of any one of the preceding Clauses 26-30, wherein the clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and/or an HCS clustering algorithm.
Clause 32: The computer readable media of any one of the preceding Clauses 26-31, wherein the set of weight and bias values is a final set of weight and bias values of the trained electronic neural network.
Clause 33: The computer readable media of any one of the preceding Clauses 26-32, wherein the instructions which, when executed by the processor, further perform operations comprising: determining whether a test subject has at least one of the disease states using a peptide sequence and binding value pair data set obtained from the test subject and the trained electronic neural network and/or the disease map.
Clause 34: The computer readable media of any one of the preceding Clauses 26-33, wherein the instructions which, when executed by the processor, further perform operations comprising: generating at least one therapy recommendation for the test subject based at least in part a determination that the test subject has the at least one of the disease states.
Clause 35: The computer readable media of any one of the preceding Clauses 26-34, wherein the instructions which, when executed by the processor, further perform operations comprising: generating at least one iteration of the disease map at a time point that differs from a time point at which the disease map was generated.
Clause 36: The computer readable media of any one of the preceding Clauses 26-35, wherein the instructions which, when executed by the processor, further perform operations comprising: monitoring an immune status measure of the population and/or an occurrence of a known disease state or an unknown disease state in the population using the disease map and the iteration of the disease map.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

What is claimed is:

1. A computer-implemented method of generating a disease map of a population, the method comprising applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.

2. The computer-implemented method of claim 1, wherein at least one of the disease states is known.

3. The computer-implemented method of claim 1, wherein at least one of the disease states is unknown.

4. The computer-implemented method of claim 1, wherein at least one of the disease states comprises an infectious disease state.

5. The computer-implemented method of claim 1, wherein the disease map comprises clusters of the disease states represented in a two or more dimensional space.

6. The computer-implemented method of claim 1, wherein the clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and/or an HCS clustering algorithm.

7. The computer-implemented method of claim 1, wherein the set of weight and bias values is a final set of weight and bias values of the trained electronic neural network.

8. The computer-implemented method of claim 1, further comprising producing the peptide sequence and binding value pair data sets from samples obtained from the reference subjects in the population.

9. The computer-implemented method of claim 1, further comprising determining whether a test subject has at least one of the disease states using a peptide sequence and binding value pair data set obtained from the test subject and the trained electronic neural network and/or the disease map.

10. The computer-implemented method of claim 9, further comprising generating at least one therapy recommendation for the test subject based at least in part on a determination that the test subject has the at least one of the disease states.

11. The computer-implemented method of claim 10, further comprising administering one or more therapies to the test subject based at least in part on the therapy recommendation for the test subject.

12. The computer-implemented method of claim 1, further comprising generating at least one iteration of the disease map at a time point that differs from a time point at which the disease map was generated.

13. The computer-implemented method of claim 12, further comprising monitoring an immune status measure of the population and/or an occurrence of a known disease state or an unknown disease state in the population using the disease map and the iteration of the disease map.

14. The disease map produced by the method of claim 1.

15. A system for generating a disease map of a population using an electronic neural network, the system comprising:
a processor; and
a memory communicatively coupled to the processor, the memory storing instructions which, when executed on the processor, perform operations comprising:
applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.

16. The system of claim 15, wherein at least one of the disease states is known.

17. The system of claim 15, wherein at least one of the disease states is unknown.

18. The system of claim 15, wherein at least one of the disease states comprises an infectious disease state.

19. The system of claim 15, wherein the disease map comprises clusters of the disease states represented in a two or more dimensional space.

20. The system of claim 15, wherein the clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and/or an HCS clustering algorithm.

21. The system of claim 15, wherein the set of weight and bias values is a final set of weight and bias values of the trained electronic neural network.

22. The system of claim 15, wherein the instructions which, when executed on the processor, further perform operations comprising:
determining whether a test subject has at least one of the disease states using a peptide sequence and binding value pair data set obtained from the test subject and the trained electronic neural network and/or the disease map.

23. The system of claim 22, wherein the instructions which, when executed on the processor, further perform operations comprising:
generating at least one therapy recommendation for the test subject based at least in part a determination that the test subject has the at least one of the disease states.

24. The system of claim 15, wherein the instructions which, when executed on the processor, further perform operations comprising:
generating at least one iteration of the disease map at a time point that differs from a time point at which the disease map was generated.

25. The system of claim 24, wherein the instructions which, when executed on the processor, further perform operations comprising:
monitoring an immune status measure of the population and/or an occurrence of a known disease state or an unknown disease state in the population using the disease map and the iteration of the disease map.

26. A computer readable media comprising non-transitory computer executable instructions which, when executed by at least one electronic processor, perform at least:
applying a clustering algorithm to a set of weight and bias values of a trained electronic neural network to generate the disease map of the population, wherein the electronic neural network has been trained on training data that comprises representations of peptide sequence and binding value pair data sets obtained from reference subjects in the population, wherein a given peptide sequence and binding value pair data set comprises peptide sequence information and peptide binding values of one or more antibodies to one or more peptides that comprises the peptide sequence information, which antibodies are from a sample obtained from a given reference subject in the population and which antibodies are indicative of one or more disease states.

27. The computer readable media of claim 26, wherein at least one of the disease states is known.

28. The computer readable media of claim 26, wherein at least one of the disease states is unknown.

29. The computer readable media of claim 26, wherein at least one of the disease states comprises an infectious disease state.

30. The computer readable media of claim 26, wherein the disease map comprises clusters of the disease states represented in a two or more dimensional space.

31. The computer readable media of claim 26, wherein the clustering algorithm comprises a Uniform Manifold Approximation and Projection (UMAP) algorithm, a Principal Component Analysis (PCA) algorithm, a hierarchical clustering algorithm, a k-means algorithm, an expectation-maximization algorithm, and/or an HCS clustering algorithm.

32. The computer readable media of claim 26, wherein the set of weight and bias values is a final set of weight and bias values of the trained electronic neural network.

33. The computer readable media of claim 26, wherein the instructions which, when executed by the processor, further perform operations comprising:
determining whether a test subject has at least one of the disease states using a peptide sequence and binding value pair data set obtained from the test subject and the trained electronic neural network and/or the disease map.

34. The computer readable media of claim 33, wherein the instructions which, when executed by the processor, further perform operations comprising:
generating at least one therapy recommendation for the test subject based at least in part a determination that the test subject has the at least one of the disease states.

35. The computer readable media of claim 26, wherein the instructions which, when executed by the processor, further perform operations comprising:
generating at least one iteration of the disease map at a time point that differs from a time point at which the disease map was generated.

36. The computer readable media of claim 35, wherein the instructions which, when executed by the processor, further perform operations comprising:
monitoring an immune status measure of the population and/or an occurrence of a known disease state or an unknown disease state in the population using the disease map and the iteration of the disease map.
